(19) United States
(12) Patent Application Publication
(10) Pub. No.: US 2016/0152566 A1
(43) Pub. Date: Jun. 2, 2016

(54) TRICYCLIC PYRROLE COMPOUNDS, THEIR PREPARATION AND USE IN MEDICAMENTS

(71) Applicant: INSTITUTO BIOMAR, S.A., Armunia-León (ES)

(72) Inventors: Antonio FERNÁNDEZ MEDARDE, Armunia-León (ES); Librada Maria CANEDO HERNÁNDEZ, Armunia-León (ES); María De Los Ángeles VINUESA-NAVARRO, Armunia-León (ES); José María SÁNCHEZ LÓPEZ, Armunia-León (ES); Belinda CALVO PELAZ, Armunia-León (ES); Marta MARTÍNEZ INSUA, Armunia-León (ES); Ana RODRÍGUEZ-QUESADA, Armunia-León (ES); Melissa CARCÍA-CABALLERO, Armunia-León (ES); Miguel-Ángel MEDINA-TORRES, Armunia-León (ES)

(21) Appl. No.: 14/903,811

(22) PCT Filed: Jul. 8, 2014
(86) PCT No.: PCT/EP2014/064641

§ 371 Date: Jan. 8, 2016

(30) Foreign Application Priority Data
Jul. 9, 2013 (EP) 13382284.1

(51) Int. Cl. C07D 209/70

(52) U.S. Cl. CPC C07D 209/70

(57) ABSTRACT

The invention refers to compounds of general formula (I)

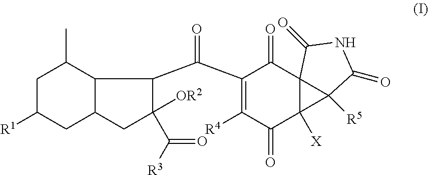

wherein the variables take the various meanings, pharmaceutical compositions containing them and their use in medicine. The compounds have shown to be useful in therapy as angiogenesis inhibitors. The compounds are isolated from the fermentation broth of a fungal strain Paraconiothyrium sp., or are derivatives thereof.

### FIELD OF THE INVENTION

The present invention relates to compounds, pharmaceutical compositions containing them and their use in medicine for use in the treatment of human diseases caused by pathological angiogenesis In particular this invention relates to new angiogenesis inhibitor compounds such as AD0157, isolated from the fermentation broth of a microorganism, a fungal strain Paraconiothyrium sp., and derivatives of these compounds.

### BACKGROUND

Angiogenesis, the process of formation of new blood vessels From other pre-existent ones, is strictly controlled by a balance of activators and inhibitors. When angiogenic growth factors are produced in excess of angiogenic inhibitors, the balance is tipped in favour of Mood vessel growth, connecting the so called “angiogenic switch”. Angiogenesis is reduced in the adult to some processes related to reproductive cycles (corpus luteum formation, endometrial vascularization, placental development), wound heating and bone repair. In all these cases, angiogenesis takes places as a transient and highly regulated process. On the contrary, a persistent and deregulated angiogenesis is an essential step in the transition of tumors from a dormant state to a malignant state.

While angiogenesis is normally a regulated process, many diseases (characterized as angiogenic diseases) are driven by persistent, unregulated angiogenesis. Ocular neovascularization has been implicated as the most common cause of blindness and is responsible for approximately twenty different eye diseases. In certain existing conditions, such as arthritis, newly formed capillary blood vessels invade the joints and destroy cartilage. The growth and metastasis of solid tumors are also dependent on angiogenesis (Cancer Res. 1986, 46, 467-473, and J. Natl. Cancer Inst. 1989, 82, 4-6).

Nowadays, angiogenesis is considered to be one of the hallmarks of cancer, playing a relevant role in tumor growth, invasion, and metastasis (Hannahan et al., Cell 144 (5):646-74, 2011). The role of the angiogenesis switch is not limited to the neoplasic diseases pathogenesis, but it has also been related to other non-neoplasic diseases including wet macular degeneration, diabetic retinopathies, diabetes, psoriasis and rheumatoid arthritis, among others. All these facts make angiogenesis inhibition an attractive target in the field of pharmacological research. A continuously increasing number of antiangiogenic therapies are being approved for the treatment of cancer, blindness, and other angiogenesis-dependent diseases, encouraging expectations in their therapeutic potential (Quesada et al., Curr. Pharm. Des., 16 (35):3932-57, 2010).

JP2008001720 discloses ansamycin antibiotics derived from microorganisms, as angiogenesis inhibitors.

US2005131061 describes a compound produced by a microorganism belonging to the genus Aspergillus having an angiogenesis inhibiting activity.

The design and development of small molecules with good antiangiogenic properties is an attractive approach for the development of new therapeutic agents, and there is a need for such molecules.

### BRIEF DESCRIPTION OF THE INVENTION

The present invention discloses novel compounds having antiangiogenic properties, and therefore useful in the treatment of diseases caused by a pathological angiogenesis process. The results in the examples clearly show that AD0157, a compound of the invention, inhibits angiogenesis in vitro and also in vivo. It inhibits the growth and induces apoptosis in tumor and endothelial cells; it inhibits endothelial lube formation on a layer of Matrigel, and decreases the endothelial proteolytic capability, in vivo, the antiangiogenic activity of this compound was shown by the chick chorioallantoic membrane (CAM). In the Matrigel plug, and in the zefrafish embryo neovascularization and zebrafish caudal fin regeneration assays. Taken together, these data indicate that the compound of the invention inhibits several essential steps of the angiogenic process.

Specifically, it is an object of the present invention a compound of general formula (I), or a pharmaceutically acceptable salt, isomer, prodrug or solvate thereof:

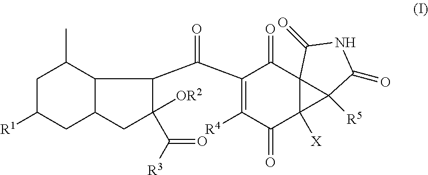

wherein R¹ Is selected from the group consisting of —COOR^{a}, —CONHR^{a}, —CONR^{a}R^{b}, —COX, —CN, —C(═O)H, —CH₂OR^{a}, —CH₂X;

R^{a} and R^{b} are independently selected from the group consisting of hydrogen, substituted or unsubstituted acyl, substituted or unsubstituted alkyl, substituted or unsubstituted alkenyl, substituted or unsubstituted alkynyl, substituted or unsubstituted aryl, substituted or unsubstituted benzyl, alkali-metal, and sugar;

X is selected from the group consisting of hydrogen and halogen;

R² is selected from the group consisting of hydrogen, substituted or unsubstituted acyl, substituted or unsubstituted alkyl, substituted or unsubstituted alkenyl, substituted or unsubstituted alkynyl, substituted or unsubstituted aryl, substituted or unsubstituted benzyl, alkali-metal, and sugar;

R³, R⁴ and R⁵ are independently selected from the group consisting of hydrogen, substituted or unsubstituted alkyl, substituted or unsubstituted alkenyl and substituted or unsubstituted alkynyl;

or a pharmaceutically acceptable salt, stereoisomer, or solvate thereof.

Another object of the invention refers to different processes for the preparation of a compound of general formula (I) as defined above, or a pharmaceutically acceptable salt, stereoisomer, or solvate thereof.

Another object of the invention refers to a medicament or pharmaceutical composition comprising at least one compound of general formula (I) as defined above, or a pharmaceutically acceptable salt, stereoisomer, or solvate thereof and at least one pharmaceutically acceptable excipient.

Another object of the invention refers to a compound of general formula (I) as defined above, or a pharmaceutically acceptable salt, stereoisomer, or solvate thereof, for use as a medicament, particularly for the treatment and/or prophylaxis of a human disease caused by a pathological angiogenesis process.

Another object of the invention refers to the use of a compound of general formula (I) as defined above, or a pharmaceutically acceptable salt, stereoisomer, or solvate thereof, in the manufacture of a medicament for the treatment and/or prophylaxis of a human disease caused by a pathological angiogenesis process.

Another object of the invention refers to a method for the treatment and/or prophylaxis of a human disease caused by a pathological angiogenesis process, the method comprising administering to the subject in need of such a treatment or prophylaxis; a therapeutically effective amount of a compound of general formula (I) as defined above, or a pharmaceutically acceptable salt, stereoisomer, or solvate thereof.

In one embodiment, said disease or condition is selected from the group consisting of cancer, ocular diseases, psoriasis, hemangiomas, arthritis, endometriosis, and atherosclerosis.

In a particular embodiment, the disease or condition is cancer.

In one embodiment, the ocular disease is selected from the group formed by diabetic and non-diabetic retinopathy, prematurity retinopathy and macular degeneration.

In another embodiment, the arthritis is selected from the group formed by osteoarthritis, rheumatoid arthritis, polyarthritis, gouty arthritis, lupus-associated arthritis, psoriasis-associated arthritis, and rheumatoid arthritis.

These aspects and preferred embodiments thereof are additionally also defined hereinafter in the detailed description, as well as in the claims.

### BRIEF DESCRIPTION OF DRAWINGS

FIG. 1. AD0157 inhibits endothelial and tumor cell growth
(A) Dose-dependent effect of AD0157 on the in vitro growth of BAE (▴), MDA-MB231 (x), HT29 (♦), HT1080 (▪) and U2OS (). Cell survival is represented as a percentage of control-cells growth in cultures containing no drug. Each point represents the mean of quadruplicates; SD values were typically lower than 10% of the mean values and are omitted for clarity.
(B) Half-maximal inhibitory concentration (IC50) values calculated from dose-response curves as the concentration of compound yielding 50% of control cell survival. They are expressed as means ±SD of three independent experiments.
(C) Half-maximal inhibitory concentration (IC50) values calculated from dose-response curves as the concentration of compound yielding 50% of control cell survival. They are expressed as means ±SD of two independent experiments, first, an experiment with single sample and second, an experiment with triplicate samples.
IC50 value for BAEC have been obtained from a experiment with single sample.

FIG. 2. Effect of AD0157 on cell morphology by Hoechst staining in endothelial and tumor cells (bar=50 μm).

FIG. 3. AD0157 inhibits endothelial cell tube formation
BAEC seeded on Matrigel formed tubes. AD0157 inhibited endothelial cell tubulogenesis in vitro in a dose-dependent manner at non-toxic doses. Cells were photographed 7 h after seeding under an inverted microscope (bar=100 μm).

FIG. 4. AD0157 inhibits the migratory potential of endothelial cell
(A) Confluent BAEC monolayers were wounded and fresh culture medium was added either in the absence or presence of the indicated concentrations of the compound. Photographs ware taken at the beginning of the assay and after 24 hours of incubation. Broken lines indicate the wound edges (bar=100 μm)
(B) The regrowth of BAEC into the cell-free area was measured after 7 h and percentages of recovered area are expressed as mean ±SD. *P<0.05 versus control (n=3).

FIG. 5. Conditioned media and cellular extracts from BAE cells (A) or HT1080 cells (B) were treated during 24 hours with the indicated concentrations of AD0157, were normalized for equal cell density and used for gelatin zymography. Graphics show the quantification of the normalized relative inhibitory affect on BAEC MMP-2 activity or HT1060 MMP-2 and MMP-9 activities. Data are given as percentage of the untreated control, and they are means ±SD of three experimental values. *P<0.05 versus control and **P<0.005 versus control.

FIG. 6. Induction of apoptosis by AD0157 on BAE cells
(A) Effect Of AD0157 on DNA fragmentation (TUNEL assay) (bar=50 μm)
(B) Effect of AD0157 on sub-G1 cell population. Results are mean ±SD of three independent experiments.
(C) Effect of AD0157 on the endothelial cells caspase-3-like activity. Results are mean ±SD of three independent experiments. *P<0.005 versus control.

FIG. 7. AD0157 Inhibits chorioallantoic membrane assay.
Methylcellulose disc containing the substance vehicle alone and methylcellulose disc containing 0.6, 0.1 and 5 nmol of AD0157. Circles show the locations of the methyl cellulose discs (bar=1 mm)

FIG. 8. Inhibition of the zebrafish neovascularization by AD0157.
Transgenic TGfli1:EGFPy1 zebrafish embryos, which show green fluorescent protein (GFP) expression in endothelial cells, were incubated without or with 5, 10 and 25 μM AD0157 (Bars represent 50 μm).

### DETAILED DESCRIPTION OF THE INVENTION

In the context of the present invention, the following terms have the meaning detailed below.

“Alkyl” refers to a straight or branched hydrocarbon chain radical containing no unsaturation (double or triple bond), and which is attached to the rest of the molecule by a single bond. Typical alkyl groups have from 1 to about 12, 1 to about 8, or 1 to about 6 carbon atoms, e.g., methyl, ethyl, n-propyl, i-propyl, n-butyl, t-butyl, n-pentyl, etc. If substituted by cycloalkyl, it corresponds to a “cycloalkylalkyl” radical, such as cyclopropylmethyl. If substituted by aryl, it corresponds to an “arylalkyl” radical, such as benzyl, benzhydryl or phenethyl. If substituted by heterocyclyl, it corresponds to a “heterocyclylaklyl” radical.

“Alkenyl” refers to a straight or branched hydrocarbon chain radical containing at least two carbon atoms and at least one unsaturation, and which is attached to the rest of the molecule by a single bond. Typical alkenyl radicals have from 2 to about 12, 2 to about 8 or 2 to about 6 carbon atoms. In a particular embodiment, the alkenyl group is vinyl, 1-methyl-ethenyl, 1-propenyl, 2-propenyl, or butenyl.

“Alkynyl” refers to a straight or branched hydrocarbon chain radical containing at least two carbon atoms and at least one carbon-carbon triple bond, and which is attached to the rest of the molecule by a single bond. Typical alkynyl radicals have from 2 to about 12, 2 to about 8 or 2 to about 6 carbon atoms. In a particular embodiment, the alkynyl group is ethynyl, propynyl (e.g. 1-propynyl, 2-propynyl), or butynyl (e.g. 1-butynyl, 2-butynyl, 3-butynyl).

“Cycloalkyl” refers to an alicyclic hydrocarbon. Typical cycloalkyl radicals contain from 1 to 4 separated and/or fused rings and from 3 to about 18 carbon atoms, preferably from 3 to 10 carbon atoms, such as cyclopropyl, cyclohexyl or adamantyl. In a particular embodiment, the cycloalkyl radical contains from 3 to about 6 carbon atoms.

“Aryl” refers to single and multiple ring radicals, including multiple ring radicals that contain separate and/or fused aryl groups. Typical aryl groups contain from 1 to 3 separated and/or fused rings and from 6 to about 18 carbon ring atoms, preferably from 6 to about 14 carbon ring atoms, such as phenyl, 1- or 2-naphthyl, biphenyl, indenyl, fenanthryl or anthracyl radical.

“Heterocyclyl” include heteroaromatic and heteroalicyclic groups containing from 1 to 3 separated and/or fused rings and from 3 to about 18 ring atoms. Preferably heteroaromatic and heteroalicyclic groups contain from 5 to about 10 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pterindinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl. Suitable heteroalicyclic groups. In the compounds of the present invention contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thoioxanyl, piperazinyl, azetidinyl, oxetanyl, thietanyl, homopiperidinyl, oxepanyl, thiepanyl, azepinyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, ditholanyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

The groups above mentioned may be substituted at one or more available positions by one or more suitable groups such as OR′, ═O, SR′, SOR′, SO₂R′, OSO₂R′, OSO₃R′, NO₂, NHR′, N(R′)₂, ═N—R′, N(R′)COR′, N(COR′)₂, N(R′)SO₂R′, N(R′)C(═NR′)N(R′)R′, N₃, CN, halogen, COR′, COOR′, OCOR′, OCOOR′, OCONHR′, OCON(R′)₂, CONHR′, CON(R′)₂, CON(R′)OR′, CON(R′)SO₂R′, PO(OR′)₂, PO(OR′)R′, PO(OR′)N(R′)R′), C₁-C₁₂ alkyl, C₃-C₁₀ cycloalkyl, C₂-C₁₂ alkenyl, C₂-C₁₂ alkynyl, aryl, and heterocyclic group, wherein each of the R′ groups is independently selected from the group consisting of hydrogen, OH, NO₂, NH₂, SH, CN, halogen, COH, COalkyl, COOH, C₁-C₁₂ alkyl, C₃-C₁₀ cycloalkyl, C₂-C₁₂ alkenyl, C₂-C₁₂ alkynyl, aryl and heterocyclic group. Where such groups are themselves substituted, the substituents may be chosen from the foregoing list.

“Halogen”, “halo” or “hal” refers to bromo, chloro, iodo or fluoro.

The term “salt” must be understood as any form of a compound used in accordance with this invention in which said compound is in ionic form or is charged and coupled to a counter-ion (a cation or anion) or is in solution. This definition also includes quaternary ammonium salts and complexes of the molecule with other molecules and ions, particularly, complexes formed via ionic interactions. The definition includes in particular physiologically acceptable salts; this term must be understood as equivalent to “pharmacologically acceptable salts” or “pharmaceutically acceptable salts”.

The term “pharmaceutically acceptable salts” in the context of this invention means any salt that is tolerated physiologically (normally meaning that it is not toxic, particularly, as a result of the counter-ion) when used in an appropriate manner for a treatment, applied or used, particularly, in humans and/or mammals. These physiologically acceptable salts may be formed with cations or bases and, in the context of this invention, are understood to be salts formed by at least one compound used in accordance with the invention—normally an acid (deprotonated)—such as an anion and at least one physiologically tolerated cation, preferably inorganic, particularly when used on humans and/or mammals. Salts with alkali and alkali earth metals are preferred particularly, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochlorid acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “solvate” in accordance with this invention should be understood as meaning any form of the compound in accordance with the invention in which said compound is bonded by a non-covalent bond to another molecule (normally a polar solvent), including especially hydrates and alcoholates, like for example, methanolate. A preferred solvate is the hydrate.

Any compound referred to herein is intended to represent such specific compound as well as certain variations or forms. In particular, compounds referred to herein may have asymmetric centres and therefore exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are enamine-imine, amide-imidic acid, keto-enol, lactam-lactim, etc.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, stereoisomers or solvates.

As used herein, the terms “treat”, “treating” and “treatment” include the eradication, removal, reversion, alleviation, modification, or control of a disease or condition after its onset.

As used herein, the terms “prevention”, “preventing”, “preventive” “prevent” and “prophylaxis” refer to the capacity of a therapeutic to avoid, minimize or difficult the onset or development of a disease or condition before is onset.

Therefore, by “treating” or “treatment” and/or “preventing” or “prevention”, as a whole, is meant at least a suppression or an amelioration of the symptoms associated with the condition afflicting the subject, where suppression and amelioration are used in a broad sense to refer to at least a reduction in the magnitude of a parameter, e.g., symptom associated with the condition being treated. As such, the method of the present invention also includes situations where the condition is completely inhibited, e.g., prevented from happening, or stopped, e.g., terminated, such that the subject no longer experiences the condition.

The compounds of the invention are suitable as pharmacologically active agents in medicaments for the prophylaxis and/or treatment of disorders or diseases related to angiogenesis.

In a particular embodiment, R¹ is selected from —COOR^{a}, —CONHR^{a}, —CONR^{a}R^{b} wherein R^{a} and R^{b} are as defined above.

In a more particular embodiment, R¹ represents —COOR^{a} more preferably —COOH or a salt thereof.

In another particular embodiment, R² is Hydrogen.

In another particular embodiment R³ is substituted or unsubstituted alkenyl, preferably unsubstituted alkenyl. In a particularly preferred embodiment R³ is 1-propenyl.

In another particular embodiment R⁴ is substituted or unsubstituted alkenyl, preferably unsubstituted alkenyl. In a particularly preferred embodiment R⁴ is 1-propenyl.

In a more preferred embodiment both R³ and R⁴ are 1-propenyl.

In another particular embodiment R⁵ is substituted or unsubstituted alkyl, preferably unsubstituted alkyl. In a more preferred embodiment R⁵ is straight or branched C1-C12 alkyl, preferably C1-C7 alkyl, more preferably C3-C6 alkyl. In a particular embodiment unsubstituted straight alkyl is preferred, especially hexyl.

In another particular embodiment X is hydrogen.

In another particular embodiment X is halogen, preferably Cl.

In additional preferred embodiments, 2 or more of the preferences described above for the different substituents are combined. The present invention is also directed to such combinations of preferred substitutions in the formula (I) above.

A particular individual compound of the invention falling under formula (I) include the compound AD0157

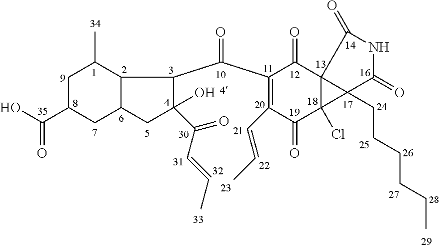 [SMILES: C/C=C/C(=O)C1(O)CC2CC(C(=O)O)CC(C)C2C1C(=O)C1=C(/C=C/C)C(=O)C2(Cl)C3(CCCCCC)C(=O)NC(=O)C23C1=O]

or a pharmaceutically acceptable salt, stereoisomer, or solvate thereof.

The compounds of general formula (I) can be isolated from the fermentation broth of a microorganism, a fungal strain Paraconiothyrium sp. HL-78-gCHSP3-B005, a culture of which has been deposited in the Colección Española de Cultivos Tipo at the Universidad de Valencia, Spain under the accession number CECT 20841. This deposit has been made under the provisions of the Budapest Treaty and all restrictions on the availability thereof to the public will be irrevocably maintained upon the granting of a patent on this application.

The microorganism was isolated from a marine chordata sample collected in Guatemala.

In a further aspect the invention is directed to a process for the preparation of a compound of formula (I) as defined above which comprises the step of isolating a compound of formula (I) above from a culture broth of a microorganism.

In a preferred embodiment the microorganism is a fungal strain, more preferably a Paraconiothyrium sp. In a particular embodiment the fungal strain is Paraconiothyrium sp. HL-78-gCHSP3-B005 which has been deposited in the Colección Española de Cultivos Tipo at the Universidad de Valencia, Spain under the accession number CECT 20841.

While the deposited strain is clearly preferred, the present invention is not restricted or limited to any particular strain or organisms. Other AD0157 producing organisms, strains or mutants are within the scope of this invention.

Paraconiothyrium sp cultured under controlled conditions in a suitable medium produces the angiogenesis inhibitor compound AD0157. This strain is preferably grown in an aqueous nutrient medium, under aerobic and mesophilic conditions.

The compounds of formula (I) of the invention can be produced by the microorganism, or a genetically modified microorganism derived from the above mentioned strain.

Alternatively, the compounds of formula (I) or can be derived from other compounds having the same basic structure, such as AD0157. Therefore, compounds of the invention can be obtained by modifying those already obtained from the natural source or by further modifying those already modified by using a variety of chemical reactions. Thus, hydroxyl groups can be acylated by standard coupling or acylation procedures, for instance by using acetic acid, acetyl chloride or acetic anhydride in pyridine or the like. Formate groups can be obtained by heating hydroxyl precursors in formic acid. Carbamates can be obtained by heating hydroxyl precursors with isocyanates. Hydroxyl groups can be converted into halogen groups through intermediate sulfonates for iodide, bromide or chloride, or directly using a sulfur trifluoride for fluorides; or they can be reduced to hydrogen by reduction of intermediate sulfonates. Hydroxyl groups can also be converted into alkoxy groups by alkylatlon using an alkyl bromide, iodide or sulfonate, or into amino lower alkoxy groups by using, for instance, a protected 2-bromoethylamine. Amido groups can be alkylated or acylated by standard alkylation or acylation procedures, for instance by using, respectively. KH and methyl iodide or acetyl chloride in pyridine or the like. Ester groups can be hydrolized to carboxylic acids or reduced to aldehyde or to alcohol. Carboxylic acids can be coupled with amines to provide amides by standard coupling or acylation procedures. When necessary, appropriate protecting groups can be used on the substituents to ensure that reactive groups are not affected. The procedures and reagents needed to prepare these derivatives are known to the skilled person and can be found in general textbooks such as March's Advanced Organic Chemistry 6th Edition 2007, Wiley Interscience.

For example R¹ can be transformed using standard Organic Chemistry reactions, such as those described below:

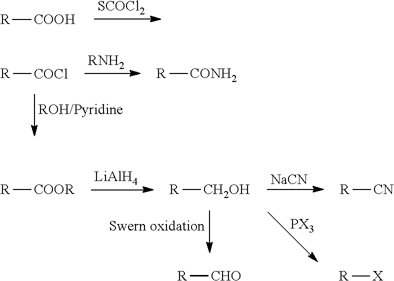

Reaction of the carboxylic acid with thionyl chloride gives the corresponding acid chloride, which can be converted in an amide by reaction with the corresponding amine. Reaction of the acid chloride with an alcohol in presence of pyridine affords the corresponding ester, which can be easily converted in the corresponding alcohol by different reduction methods. Swern oxidation of the alcohol affords the corresponding aldehyde. Reaction of the alcohol with NaCN gives the corresponding nitrile, and finally the corresponding halide can be obtained by reaction of the alcohol with PX3.

In another embodiment, the introduction of R² in the molecule can be achieved by any of the classic methods of hydroxyl group protection (via formation of ether, ester, etc).

Modifications in the alkenyl groups R³ and R⁴, can be achieved by double bond hydrogenation, epoxidation or different addition reactions to the double bond and further modifications.

It is also an object of the invention to provide medicaments or pharmaceutical compositions comprising at least one compound of general formula (I) as defined above, or a pharmaceutically acceptable salt, stereoisomer, or solvate thereof, and at least one pharmaceutically acceptable excipient.

The term “excipient” refers to components of a drug compound other than the active ingredient (definition obtained from the European Medicines Agency—EMA). They preferably include a “carrier, adjuvant and/or vehicle”. Carriers are forms to which substances are incorporated to improve the delivery and the effectiveness of drugs. Drug carriers are used in drug-delivery systems such as the controlled-release technology to prolong in vivo drug actions, decrease drug metabolism, and reduce drug toxicity. Carriers are also used in designs to increase the effectiveness of drug delivery to the target sites of pharmacological actions (U.S. National Library of Medicine, National Institutes of Health). Adjuvant is a substance added to a drug product formulation that affects the action of the active ingredient in a predictable way. Vehicle is an excipient or a substance, preferably without therapeutic action, used as a medium to give bulk for the administration of medicines (Stedman's Medical Spellchecker© 2006 Lippincott Williams & Wilkins). Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, disgregants, wetting agents or diluents. Suitable pharmaceutical carriers are described in “Remington's Pharmaceutical Sciences” by E. W. Martin. The selection of these excipients and the amounts to be used will depend on the form of application of the pharmaceutical composition.

The pharmaceutical compositions in accordance with the invention can be adapted in order to be administered by any route of administration, be it orally or parenterally, such as pulmonarily, nasally, rectally and/or intravenously. Therefore, the formulation in accordance with the invention may be adapted for topical or systemic application, particularly for dermal, subcutaneous, intramuscular, intra-articular, intraperitoneal, pulmonary, buccal, sublingual, nasal, percutaneous, vaginal, oral or parenteral application.

Suitable preparations for oral applications are tablets, pills, chewing gums, capsules, granules, drops or syrups. Suitable preparations for parenteral applications are solutions, suspensions, reconstitutable dry preparations or sprays.

The pharmaceutical composition of the invention may be formulated as deposits in dissolved form or in patches, for percutaneous application. Skin applications include ointments, gels, creams, lotions, suspensions or emulsions.

Another aspect of the invention is a method for the treatment and/or prophylaxis of a human disease caused by a pathological angiogenesis process, the method comprising administering to the subject in need of such a treatment or prophylaxis a therapeutically effective amount of a compound of formula (I) as defined above, or a pharmaceutically acceptable salt, stereoisomer, or solvate thereof.

The human disease caused by a pathological angiogenesis process in the context of this invention is preferably selected from cancer, ocular diseases, psoriasis, hemangiomas, arthritis, endometriosis, and atherosclerosis.

The term “cancer” includes both primary and metastatic solid tumors and carcinomas of, for example, the breast, colon, rectum, lung, oropharynx, hypopharynx, esophagus, stomach, pancreas, liver, gallbladder, bile ducts, small intestine, urinary tract including kidney, bladder and urothelium, female genital tract including cervix, uterus, ovaries, choriocarcinoma, and gestational trophoblastic disease, male genital tract including prostate, seminal vesicles, testes, and germ cell tumors, endocrine glands including thyroid, adrenal, and pituitary, skin including hemangiomas, melanomas, sarcomas arising from bone or soft tissues including Kaposi's sarcoma, tumors of the brain, nerves, and eyes, meninges including astrocytomas, gliomas, glioblastomas, retinoblastomas, neuromas, neuroblastomas. Schwannomas and meningiomas, solid tumors using from hematopoietic malignancies including leukemias and chloromas, plasmacytomas, plagues, tumors of mycosis fungoides, cutaneous T-cell lymphoma/leukemia, lymphomas including Hodgkin's and non-Hodgkin's lymphomas. The term “ocular diseases” refers in this text to diseases related to corneal or retinal neovascularization/angiogenesis, including diabetic retinopathy, retinopathy of prematurity, corneal graft rejection, retrolental fibroplasia, nonvascular glaucoma, rubeosis, retinal neovascularization due to macular degeneration, hypoxia, abnormal neovascularization conditions of the eye.

In a preferred embodiment of this invention the treated ocular disease is selected from diabetic and non-diabetic retinopathy, prematurity retinopathy, neovascular glaucoma and macular degeneration.

The term “corneal or retinal neovascularization” relates to the formation of new vessels invading the cornea or appearing in the retina and which can cause visual alterations and blindness, such as retinopathy or macular degeneration for example. The term “retinopathy” refers to a non-inflammatory damage to the retina of the eye, the retinopathies include diabetic and non-diabetic retinopathy and prematurity retinopathy. Particularly “diabetic retinopathy” refers to a retinopathy caused by complications of diabetes mellitus. The term “prematurity retinopathy” relates to an eye disease that affects prematurely-born babies. The term “macular degeneration” relates to loss of vision in the centre of the visual field, i.e. the macula.

The term “psoriasis” relates to and includes diseases mediated by the immune system affecting the skin and joints. When it affects the skin, it normally appears in the form of raised, flaky red patches called plaques as a result of an uncontrolled angiogenesis, epidermal cell proliferation and localized chronic inflammation. The term “hemangioma” relates to a vascular tumor occurring during childhood. The pathogenesis of hemangioma formation involves increased angiogenesis.

The term “arthritis” relates to a frequently chronic illness causing stiffness, pains and occasionally swelling of the joints and includes osteoarthritis, rheumatoid arthritis, polyarthritis, gouty arthritis, lupus-associated arthritis, psoriasis-associated arthritis and the like. In a preferred embodiment the treated arthritis is rheumatoid arthritis.

The term “endometriosis” relates to the growth of the endometrium outside the uterus, in various sites throughout the pelvis or in the abdominal wall causing symptoms such as pelvic pain, adnexal mass, dysmenorrhea, dysuria and infertility.

The term “atherosclerosis” relates to a disease affecting the arteries wherein vascular wall constrictions and irregularities are formed which hinder blood circulation and make the suitable perfusion of organs and tissues difficult.

Generally an effective administered amount of a compound used in the invention will depend on the relative efficacy of the compound chosen, the seventy of the disorder being treated, or the age, weight or mode of administration. However, active compounds will typically be administered once or more times a day, for example 1, 2, 3 or 4 times daily, with typical total dally doses in the range of from 0.1 to 500 mg/kg/day.

In a particular embodiment, the compound of Formula (I) and pharmaceutical compositions of this invention may be used together with other drugs to provide a combined therapy. The other drugs may form part of the same composition, or be provided as a separate composition for administration at the same time or at different time.

In one embodiment, the additional drug to provide a combination therapy is another pharmaceutical anti-angiogenic agent. Said other anti-angiogenic agent may be selected from anti-VEGF agents, such as monoclonal antibodies such as bevacizumab (Avastin), antibody derivatives such as ranibizumab (Lucentis), or antibody fragments such as Fab IMC 1121 or F200 Fab or orally-available small molecules that inhibit the tyrosine kinases stimulated by VEGF such as lapatinib (Tykerb), sunitinib (Sutent), sorafenib (Nexavar), axitinib, and pazopanib; anti-FGF agents, such as suramin and its derivatives, pentosan, polysulfate, cediranib, pazopanib, or BIBF 1120); anti-EGF agents, such as cetuximab, gefitinib or erlotinib and anti-HGF agents, such as ARQ197, JNJ-38877605, PF-04217903, SGX523, NK4, or AMG102; and antiangiogenic polypeptides such as angiostatin, endostatin, anti-angiogenic anti-thrombin III or sFRP-4.

As “combination administration” or “combined therapy” it is understood that the compound of formula (I) according to the invention is administered jointly or separately, simultaneously, at the same time or sequentially with another active agent, useful in the treatment and/or prophylaxis of human diseases caused by pathological angiogenesis process.

By “simultaneously”, within the meaning of the present invention is meant an administration of the at least two active agents by the same route and at the same time or at substantially the same time.

By “separately”, within the meaning of the present invention is meant in particular an administration of the at least two active agents at the same time or at substantially the same time by different routes.

By “sequentially”, is meant administration of at least two active agents at different times, the administration route being identical or different. More particularly by an administration method is meant according to which the whole administration of one of the active ingredients is carried out before administration of the other or others commences. It is thus possible to administer one of the active ingredients over several months before administering the other active ingredient or ingredients. There is no simultaneous treatment in this case. An alternate administration of each active ingredient over several weeks can also be envisaged.

Having described the present invention in general terms; it will be more easily understood by reference to the following examples which are presented as an illustration and are not intended to limit the present invention.

### EXAMPLES

### Example 1

### Identification of Producing Organism

Taxonomic studies of the strain HL-78-gCHSP3-B005 are summarised as follows:

### Culture Characteristics

Colonies reach 7 cm diameter in ten days at 25° C. on potato dextrose agar in a culture chamber that maintains a humidity of 42%.

### Colony Characteristics

Colonies growing fast, white to light brown, felt texture, becoming dark brown in concentric areas of submerged hiphae.

### Microscopy

Brown picnidia approx. 100 □μm in size, grouped around several areas of the external part of the colony. Simple conidiogenic cells, producing brown, septate, cylindrical-round ended, 8×4 μm, conidia.

Taxonomical determination was confirmed after a sequencing analysis of ITS1-5.8S-ITS2 ribosomal DNA region. Sequence shows a similarity percentage of 95% with the sequence of Paraconiothyrium variable. Conidia characteristics and low similarity value does not confirm species level, but allows to identify the isolate as Paraconiothyrium sp.

The optimal temperature for growth on solid media is 24-28° C. The pH range for growth is between 5 to 7. Growth was best with glucose and starch. Other carbon sources such as flour, glycerol, and dextrose can also be used.

Based on the preceding characteristics the culture has been determined as Paraconiothyrium sp. The fungal strain Paraconiothyrium sp. HL-78-gCHSP3-B005, was deposited in the Colección Española de Cultivos Tipo at the Universidad de Valencia, Spain under accession number CECT 20841.

### Example 2

### Production Stage

Paraconiothyrium sp. HL-78-gCHSP3-B005 when cultured under controlled conditions in a suitable medium produces the angiogenesis inhibitor compound AD0157. This strain is grown in an aqueous nutrient medium, under aerobic and mesophilic conditions, preferably between 24 and 28° C. at a pH 5.0 to 7.0.

A description of the process is as follows:

Stock culture. A pure culture of Paraconiothyrium sp. HL-78-gCHSP3-B005 was kept frozen at −70° C. in 20% glycerol.

Preparation of inoculum. A well grown agar culture was used to inoculate 40 ml of seed medium containing 2% oat meal, 2% malt extract, 0.0% KH₂PO₄. 0.005% MgSO₄, and tap water in 250 ml shake flasks and cultured at 24° C. on a rotary shaker at 200 rpm. The flasks were incubated 48 hours in the dark, and used as a first stage inoculum.

Fermentation. 250 ml of the same medium in 2 L Erlenmeyer flasks were inoculated with 10% of the first stage inoculum. The fermentation was carried for 7 days at 24° C. on a rotary shaker at 200 rpm in the dark.

Production of this compound can be monitored by HPLC or any other method with enough sensitivity.

### Isolation

Fermentation broth (4.5 L) of fungus HL-78-gCHSP3-B005 was filtered through Celite and the mycelial cake extracted twice with 2 L of a mixture of EtOAc/MeOH (3:1). The resultant suspension was filtered and partitioned between EtOAc and water. The organic layer was taken to dryness and the crude extract (9.31 g) was fractionated by VFC (vacuum flash chromatography) on silica gel, eluted with a stepwise gradient of hexane/EtOAc/MeOH. Fractions containing Compound I (eluted with EtOAc/MeOH 9:1, 225 mg) were applied to a silica gel column and flash-chromatographied by elation with a CHCl₃/MeOH gradient. Fractions containing Compound I (eluted with CHCl₃/MeOH 93:7, 83 mg) were finally purified by semipreparative reversed-phase HPLC, affording 23 mg of pure Compound I.

HPLC analysis is performed at room temperature using an analytical Symmetry C18 column (5 μm, 3.9×150 mm) and as a mobile phase gradient from 50% MeOH/H₂O (1% formic acid) to 100% MeOH in 20 min and 100% MeOH 10 minutes more, a flow rate of 0.7 mL/min, and plotted at 220 nm, in this conditions the retention time for Compound I is 17.90 min.

### Example 4

### Structural Determination

Compound I has a molecular formula of C₃₄H₄₀ClNO₉ established by APCI and API-ES mass spectra (pseudomolecular ion at m/z of 642 [M+H]+ and an isotopic peak at m/z of 644 with a ratio of 3:1), ¹³C NMR, and DEPT data. The compete assignments of ¹H and ¹³C NMR spectra of compound I, were finally established by 2D NMR experiments (COSY, HSQC and HMBC). Its spectroscopic data are shown in table 1:

TABLE 1
¹H and ¹³C NMR Spectral Data of AD0157 [δ (ppm), J_{HH} (Hz); CD₃OD]

| | ¹³C | | |
|---|---|---|---|
| Position | (δ) | ¹H (δ) | HMBC |
| 1 | 36.8 | 1.48 (1H, d, Hz) | |
| 2 | 51.4 | 1.83 (1H, d, 10.5 Hz) | C1, C2, C6, C7, C10 |
| 3 | 61.2 | 4.10 (1H, d, 10.5 Hz) | C1, C2, C4, C10, C30 |
| 4 | 86.2 | | |
| 5 | 45.4 | 1.46 (1H, m) | C4, C6, C30 |
| | | 2.05 (1H, m) | C2, C3, C6, C30 |
| 6 | 41.8 | 1.42 (1H, m) | C5 |
| 7 | 33.4 | 1.20 (1H, m) | C2, C6, C35 |
| | | 2.06 (1H, m) | C2, C6 |
| 8 | 43.3 | 2.40 (1H, m) | C34 |
| 9 | 38.3 | 1.22 (1H, m) | C1, C7, C8, C34, C35 |
| | | 1.93 (1H, m) | |
| 10 | 195.7 | | |
| 11 | 145.4 | | |
| 12 | 186.4 | | |
| 13 | 49.7^{a} | | |
| 14 | 168.6 | | |
| 15-NH | 10.4^{b} | (1H, br s) | C13, C14, C16, C17 |
| 16 | 171.9 | | |
| 17 | 48.0^{a} | | |
| 18 | 53.9^{a} | | |
| 19 | 191.1 | | |
| 20 | 148.2 | | |
| 21 | 119.0 | 6.52 (1H, dq, 16.0, 1.7 Hz) | C11, C18, C19 |
| 22 | 147.2 | 7.48 (1H, dq, 16.0, 7.0 Hz) | C19 |
| 23 | 19.6 | 2.02 (3H, dd, 7.0, 1.7 Hz) | C20, C21 |
| 24 | 21.7 | 2.06 (1H, m) | C13, C16, C18, C25, C26 |
| | | 2.27 (1H, ddd, 13.8, 11.7, | C13, C16, C17, C25, C26 |
| | | 4.23 Hz) | |
| 25 | 27.3 | 1.05 (1H, m) | C26, C27 |
| | | 1.71 (1H, m) | C26, C27 |
| 26 | 29.1 | 1.28 (2H, m) | C27 |
| 27 | 31.2 | 1.26 (2H, m) | C25, C28, C29 |
| 28 | 22.3 | 1.28 (2H, m) | C27, C29 |
| 29 | 13.2 | 0.92 (3H, t, 7.1 Hz) | C27, C28 |
| 30 | 201.6 | | |
| 31 | 125.0 | 6.68 (1H, dq, 15.4, 1.6 Hz) | C30 |
| 32 | 145.7 | 6.92 (1H, dq, 15.4, 6.8 Hz) | C30 |
| 33 | 17.6 | 1.90 (3H, dd, 6.8, 1.6 Hz) | C30, C31, C32 |
| 34 | 20.8 | 0.81 (3H, d, 6.6 Hz) | C1, C2, C9 |
| 35 | 178.3 | | |
| 4′-OH | | 3.84^{b} (1H, s) | C3, C4, C5, C30 |

^{a}interchangeable signals
^{b}Acetone-d6 spectra

### Biological Activity

Cell culture media were purchased from Biowhittaker (Wakersville, Md. USA). Fetal bovine serum (FBS) was a product of Harlan-Seralab (Belton, UK). Matrigel was purchased from Becton-Dickinson (Bedford, Mass., USA. Supplements and other chemicals not listed in this section were obtained from Sigma Chemicals (St. Louis, Mo., USA). Plastics for cell culture were supplied by NUNC (Rosklide, Denmark). Fertilised chick eggs were obtained from Granja Santa Isabel (Córdoba, Spain).

Bovine aortic endothelial (BAE) ceils were maintained in Dulbecco's modified Eagle's medium (DMEM) containing glucose (1 g/L), glutamine (2 mM), penicillin (50 IU/mL), streptomycin (0.05 mg/mL), and amphotericin (1.25 mg/L) supplemented with 10% FBS (DMEM/10% FBS). Human fibrosarcoma HT1080 cells were maintained in DMEM containing glucose (4.5 g/L), glutamine (2 mM), penicillin (50 IU/mL), streptomycin (0.05 mg/mL), and amphotericin (1.25 mg/L) supplemented with 10% FBS. Human colon adenocarcinoma HT29 and human osteosarcoma U2-OS cells were maintained in McCoy's 5A medium containing glutamine (2 mM), penicillin (50 IU/mL), streptomycin (0.05 mg/mL), and amphotericin (1.25 mg/L) supplemented with 10% FBS. Human breast cancer carcinoma MDA-MB-231 cells were maintained in RPMI1640 medium containing glutamine (2 mM) (penicillin (50 IU/mL), streptomycin (0.05 mg/mL), and amphotericin (1.25 mg/L) supplemented with 10% FBS.

Statistical analysis: All data are expressed as means ± standard deviation (SD). One-tailed Student's t test was used for evaluations of pairs of means, to establish which groups differed from the control group. P<0.05 was considered to be statistically significant.

### Example 5

### AD0157 Inhibits the Growth of Endothelial and Tumor Cells

The 3-(4,5-dimethylthiazol-2-yl)-2,5 diphenyltetrazolium bromide (MIT; Sigma Chemical Co., St. Louis, Mo.) dye reduction assay in 96-well microplates was used. The assay is dependent on the reduction of MTT by mitochondrial dehydrogenases of viable cell to a blue formazan product, which can be measured spectrophotometrically.

3×10³ BAE cells in a total volume of 100 μl of complete medium were incubated in each well with serial dilutions of the compound AD0157 obtained as explained above. After 3 days of incubation in the dark (37° C., 5% CO₂ in a humid atmosphere), 10 μl of MTT (5 mg/ml in PBS) was added to each well and the plate was incubated for further 4 h (37° C.). The resulting formazan was dissolved in 150 μl of 0.04 N HCl-2 propanol and read at 550 nm. All determinations were carried out in triplicate, independent experiments. IC₅₀ values were calculated as those concentrations of AD0157 yielding 50% cell survival, taking the values obtained for control as 100%.

For Hoechst staining experiments, cells were seeded on 8-well chamber slides and grown to sub-confluence. After treatments for 14 h with the indicated concentrations of the compound in complete medium, cells were washed (PBS) and fixed (formalin solution, Sigma). Chamber slides were stained with Hoechst, mounted (DAKO Cytomation Fluorescent Mounting Medium, DAKO), and observed under a fluorescence microscope (Leica, TCS-NT).

Angiogenesis involves local proliferation of endothelial cells. We investigated the ability of AD0157 to inhibit the proliferation of actively growing endothelial and tumor cells. As shown in FIG. 1, AD0157 inhibited the growth of cultured BAE cells with a IC₅₀ value of 10.9 μM for subconfluent BAE cells stimulated to grow with 10% FBS. Data obtained with HT1080 fibrosarcoma, HT29 colon adenocarcinoma, MDAMB231 breast carcinoma and U2OS osteosarcoma cell lines show that AD0157 is not a specific inhibitor of the endothelial cell growth, since the IC50 values of tumor cells ware in the same range of concentrations than that of BAEC.

Additionally, data obtained with A549 lung carcinoma, HCT-116 colon carcinoma, PSN1 pancreatic adenocarcinoma and T98G glioblastoma cell lines show that AD0157 is not a specific inhibitor of the endothelial cell growth, since the IC50 values of tumor cells were in the same range of concentrations than that of BAEC.

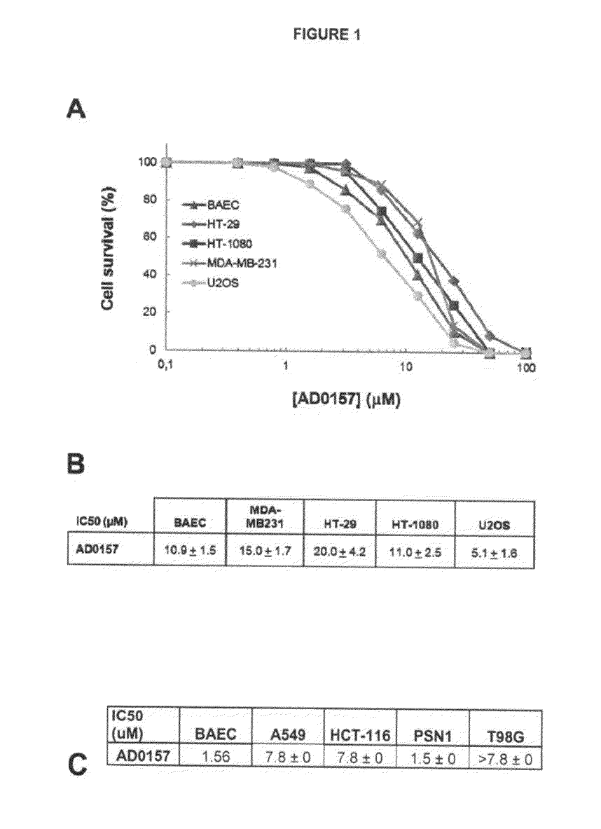

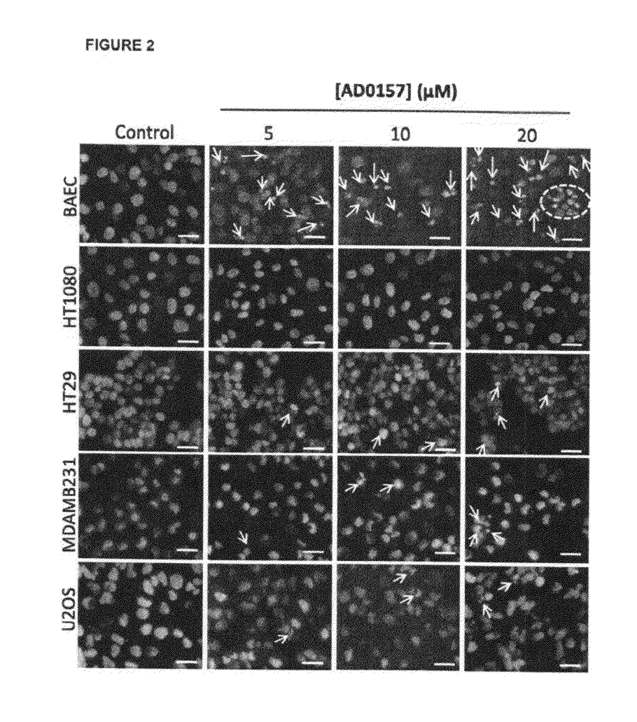

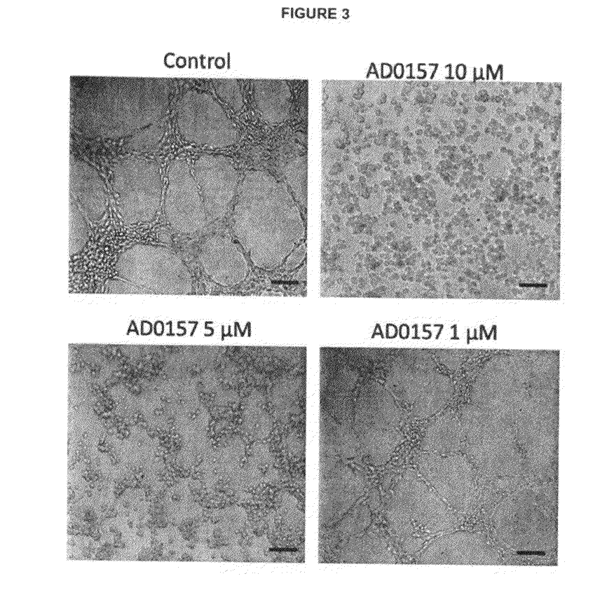

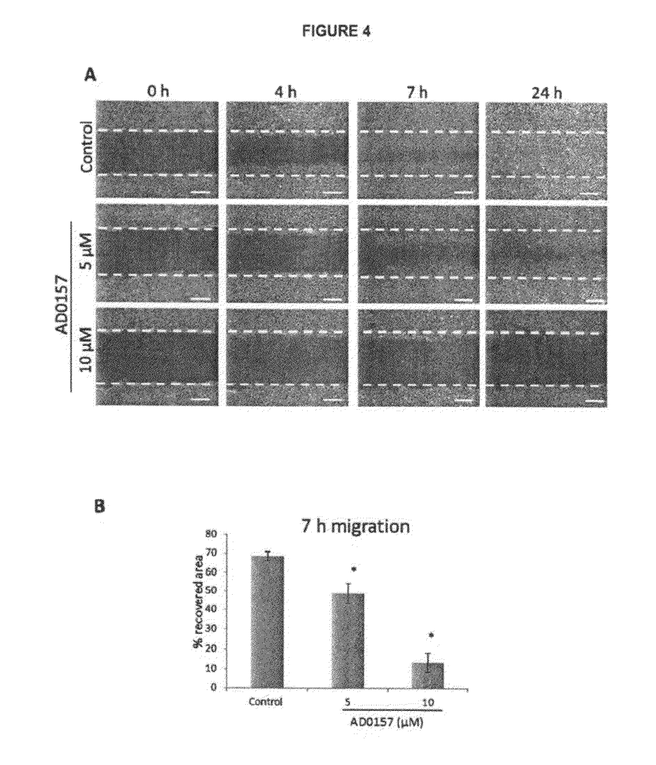

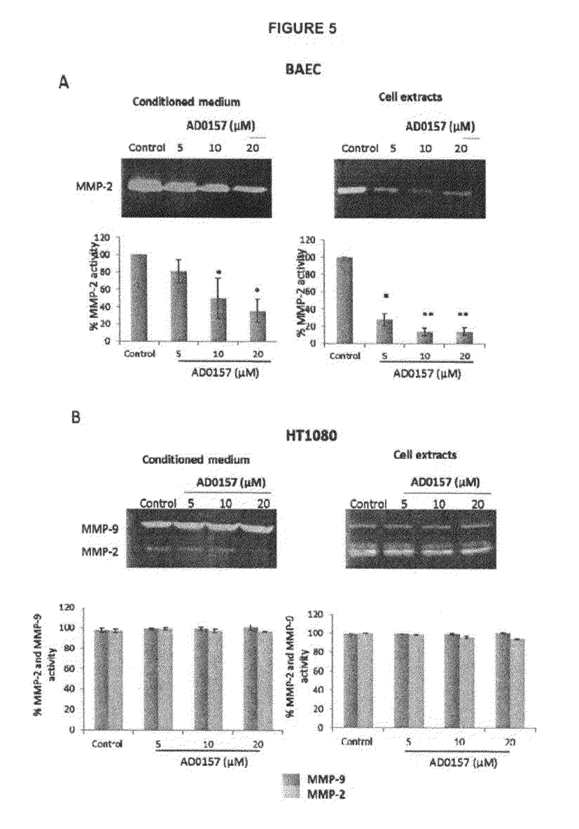

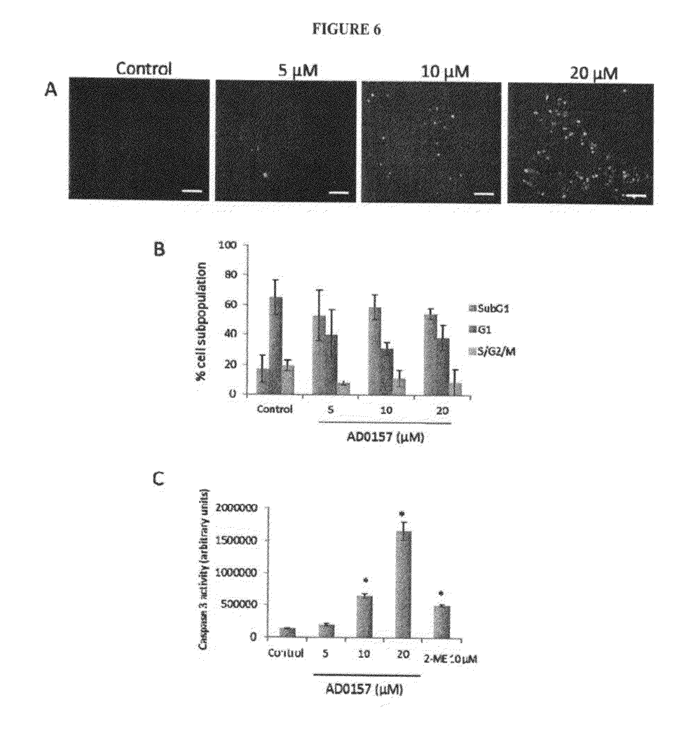

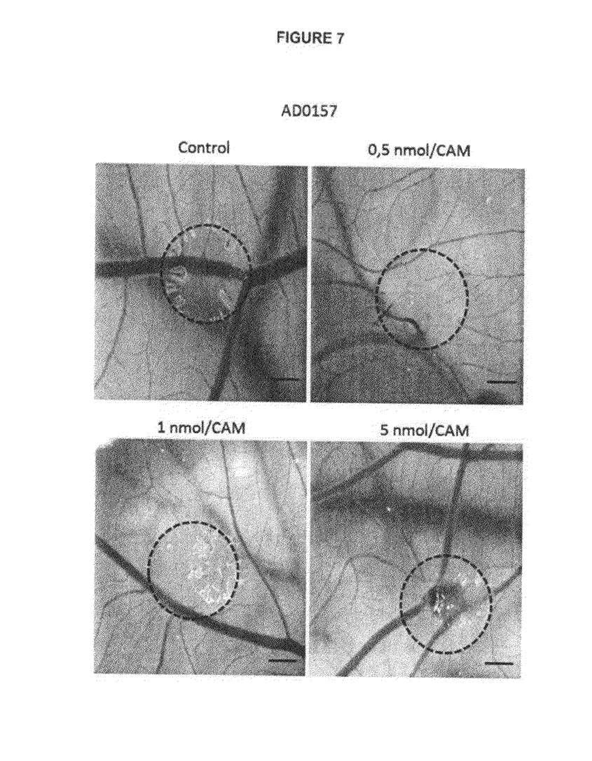

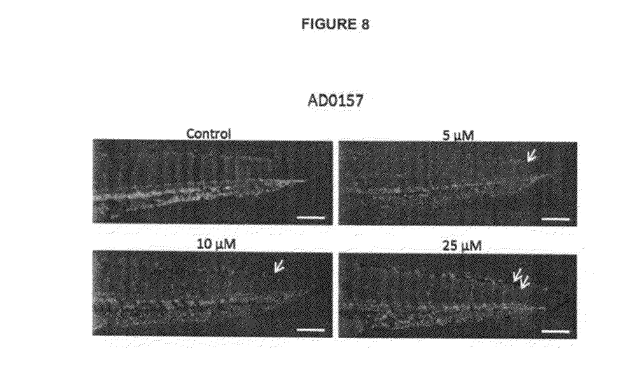

As a first approach to determine whether the growth inhibitory activity of AD0157 could be, at least in part, due to the induction of apoptosis, nuclear morphology was investigated after treatment of endothelial and tumor cells with different concentrations of these compound. FIG. 2 shows that AD0157, at concentrations above 5 μM, induced chromatin condensation in endothelial and most types of tumor cells.

### Example 6

### AD0157 Inhibits the Capillary Tube Formation by Endothelial Cells

Matrigel (50 μL of about 10.5 mg/mL) at 4° C. was used to coat each well of a 96-well plate and allowed to polymerise at 37° C. for a minimum of 30 min. 5×10⁴ BAE cells were added with 200 μL DMEM. Finally, different amounts of the Compound AD0157 were added and incubated at 37° C. in a humidified chamber with 5% CO₂. After incubation for 7 h, cultures were observed (40× and 100× magnifications) and photographed with a NIKON inverted microscope DIAPHOT-TMD (Nikon, Tokyo, Japan). Each concentration was tested in duplicate, and two different observers evaluated the inhibition of tube formation. To check the viability of endothelial cells after the treatment with the compounds in this assay, BAE cells were incubated in 96-well plate in the same conditions employed for the tube formation assay. After 7 h, cell viability in comparison to control untreated cells was determined by the addition of MTT essentially as described for the cell growth assay.

The final event during angiogenesis is the organization of endothelial cells in a three-dimensional network of tubes. As shown in FIG. 3, in vitro, endothelial cells plated on Matrigel align themselves, forming cords that are already evident a few hours after plating. Complete inhibition of endothelial morphogenesis on Matrigel was obtained at 5 μM AD0157 and partial inhibition was obtained at 1 μM AD0157 for BAECs. The treatment with AD0157, at the concentrations used to inhibit the differentiation of BAE cells, did not affect the viability of those endothelial cells alter 7 h (results not shown).

3×10⁴ BAE cells, were cultured with Cytodex beads, and allowed to adhere to them. The beads were added in 200 μL DMEM per well to a 96-well plate that has been previously prepared as follows: different amounts of the compound AD0157 were dispensed in a 96 well plate. Matrigel (50 μL) at 4° C. was used to coat each well of and allowed to polymerise at 37° C. for a minimum of 30 min. After polymerization, the beads carrying the endothelial cells were added to the wells. The plate was incubated at 37° C. in a humidified chamber with 5% CO₂. After incubation for 48 h, cultures were observed using an optical microscope. Each concentration was tested in triplicate. Inhibition of tube formation was obtained at 9.3 uM.

### Example 7

### AD0157 Inhibits the Migratory Capability of Endothelial Cells

The migratory activity of BAEC was assessed using a wounding migration assay. Confluent monolayers in 6-well plates were wounded with pipet tips following two perpendicular diameters, giving rise to two acellular 1-mm-wide lanes per well. After washing, cells were supplied with 1.5 mL complete medium in the absence (controls) or presence of different concentrations of AD0157. At different times of incubation in the dark, plates were observed under microscope and wounded areas were photographed. Photos were taken from the same areas as those recorded at zero time. The amount of migration at 7 h and 24 h was determined by image analysis in both controls and treated wells and normalized with respect to their respective values at zero time, using the NIH Image 1.6 software.

Angiogenesis involves the acquisition by endothelial cells of the capability to migrate through extracellular matrix. To investigate the effect of AD0157 on endothelial cell migration, a wound-healing assay with BAE cells was used. Our data indicate that AD0157 produced a dose-dependent inhibition of the migratory capability of endothelial cells at 7 and 24 hours of migration (FIG. 4).

### Example 8

### AD0157 Inhibits the Proteolytic Capability of Endothelial Cell

BAE or HT1080 cells were grown in 6-well plates at 75% subconfluency in 6-well plates, medium was aspirated, cells were washed twice with phosphate-buffered saline (PBS) and each well received 1.5 ml of DMEM/0.1% BSA containing 200 KIU of Aprotinin/mL. Additionally, some wells received the indicated concentration of AD0157. After 24 h of incubation, conditioned media were collected. To obtain the cell extracts, the cells were washed twice with PBS and harvested by scrapping into 0.5 mL of 0.2% Triton X-100 in 0.1 M Tris/HCl containing 200 KIU aprotinin/mL. Media were centrifuged at 1000×g and 4° C. for 20 min, and used for zymography. Duplicates were used to determine cell number with a Coulter counter.

The gelatinolytic activity of MMP-2 delivered to the conditioned media and cell extracts were detected in gelatinograms. Samples were subjected to non-reducing SDS/PAGE as above but with gelatin (1 mg/mL) added to the 10% resolving gel. After electrophoresis, gels were washed twice with 50 mM Tris/HCl, pH 7.4, supplemented with 2% Triton X-100, and twice with 50 mM Tris/HCl, pH 7.4. Each wash was for 10 min and with continuous shaking. After the washes, the gels were incubated overnight at 37° C. immersed in a substrate buffer (50 mM Tris/HCl, pH 7.4, supplemented with 1% Triton X-100, 5 mM CaCl₂, and 0.02% Na₃N). Then, the gels were stained with Commassie blue R-250 and the bands of gelatinase activity could be detected as non-stained bands in a dark, stained background.

Quantitative analysis of zymograms and gelatinograms was performed with the Scion Image 4.0.3.2 Program.

The capability to degrade the basement membrane and, in general, to remodel the extracellular matrix is essential in angiogenesis. Gelatin zymography of conditioned media and cellular extracts of AD0157-treated BAEC (FIG. 5A) shows that AD0157 inhibited MMP-2 secretion by endothelial cells. Whereas BAE cells only express MMP-2, HT1080 cells express both gelatinases: MMP-2 and MMP-9. Our results show that no effect on MMP-2 and MMP-9 levels in the HT1080 tumor cells conditioned media and cellular extracts was observed after treatment with AD0157 (FIG. 5B).

### Example 9

### AD0157 Induces Apoptosis in Endothelial and Tumor Cells by a Caspase-Dependent Mechanism

For DNA fragmentation studies, cells were grown to 75% confluency on 8-well Falcon 8 humidified chamber slides and incubated for 14 h with or without the indicated concentrations of AD0157 in complete medium. The TUNEL (terminal deoxynucleotidyl transferase mediated dUTP-biotin nick end-labeling) assay was performed with the use of the In Situ Cell Death Detection Kit (Roche Diagnostics, Barcelona, Spain), according to the manufacturer's instructions.

Cell cycle analysis was performed as follows: After treatment (14 h) with the indicated concentrations of AD0157 in complete medium, attached and unattached treated and control BAECs were harvested, washed (PBS), and fixed (70% ethanol, 1 h on ice). Pelleted cells were incubated (1 h protected from light) with RNase-A (0.1 mg/mL) and propidium iodide (40 μg/mL) during 1 h with shaking and protected from light. Percentages of sub-G1 populations were determined using a MoFlo Dakocytomation cytometer.

For the determination of Caspase 3/7 activity, BAECs were plated in 96-well plates (13000 cells/well) and treated for 14 hours with or without different concentrations of AD0157 in complete medium. Then, Caspase-Glo® 3/7 reagent (Promega Biotech Ibérica, Madrid, Spain), was added to wells, according to the manufacturer's instructions and the luminescence was recorded at thirty minutes with a GLOMAX 96 microplate luminometer. The assay provides a proluminescent caspase-3/7 DEVD-aminoluciferin substrate and a proprietary thermostable luciferase in a reagent optimized for caspase-3/7 activity, luciferase activity and cell lysis.

The induction of endothelial apoptosis is a common mechanism exhibited by a number of angiogenesis inhibitors. To confirm our previous observation that AD0157 could induce apoptosis in endothelial cells. TUNEL assay was used to detect the DNA fragmentation induced by the compound (FIG. 6A). Flow cytometric analysis of cell cycle after incubation with different doses of the compound was performed after propidium iodide staining. Results snowed that AD0157 significantly increased apoptotic sub-G1 cells at different doses (FIG. 6B). Caspases activation plays a central role in the induction of apoptosis. To determine whether caspases were activated as a result of the treatment with the compound, we used a caspase-3/7 substrate DEVD-AMC, which is cleaved to a fluorescent product by caspase-3 and other caspases with similar substrate cleavage sequences. As shown in FIG. 6C, the “effector caspase-3” was significantly activated in a dose-dependent pattern in BAE cells after treatment with AD0157. As a positive control of caspase activation, 10 μM 2-methoxyestradiol (2-ME) was used.

### Example 10

### AD0157 Inhibit in vivo Angiogenesis in the Chick Chorioallantoic Membrane Assay

Fertilised chick eggs were incubated horizontally at 38° C. in a humidified incubator, windowed by day 3 of incubation, and processed by day 8. The tested compound stock solution was added to a 1.2% solution of methylcellulose in water, and 10 μL drops of this solution were allowed to dry on a teflon-coated surface in a laminar flow hood. Then, the methylcellulose discs were implanted on the CAM, the eggs were sealed with adhesive tape and returned to the incubator for 48 h. Negative controls were always made with DMSO mixed with the methylcellulose. After the reincubation, the CAM was examined under a stereomicroscope. The assay was scored as positive when two independent observers reported a significant reduction of vessels in the treated area.

The CAM assay was used to determine the ability of the compounds to inhibit angiogenesis in vivo. In controls, blood vessels formed a dense and spatially oriented, leaf-like branching network composed by vascular structures of progressively smaller diameter as they branch (FIG. 7). Table 1 summarizes the evaluation of the in vivo inhibition of angiogenesis in the CAM assay by AD0157. As shown in that table, treatment with AD0157 caused a dose dependent anti-angiogenic effect, which is maintained as low as 0.5 nmol per CAM, where 83% of the eggs scored positive.

AD0157 exhibited a higher activity in the CAM assay, with 100% total inhibitions at 1 nmol/CAM. This effect were observed as an inhibition of the in growth of new vessels in the area covered by the methylcellulose disks and a centrifugal growth of the peripheral vessels (relative to the position of the disk), which seemed to avoid the treated area, where a decreased vascular density could be observed (FIG. 7).

TABLE 1
Inhibition of CAM angiogenesis by AD0157

| Dose (nmol/CAM) | Positive/total | % Inhibition |
|---|---|---|
| 0 | 0/12 | 0 |
| 0.1 | 1/5 | 20 |
| 0.5 | 5/6 | 83 |
| 1 | 6/6 | 100 |
| 5 | 6/6 | 100 |

### Example 11

### AD0157 Inhibits in vivo Angiogenesis in Zebrafish Embryo and Fin Assays

Zebrafish embryos were generated by natural pairwise mating and maintained in embryo water at 28.5° C. Transgenic Fli-EGFP fish (TGfli1:EGFPy1) had a label vasculature with the green fluorescent protein and were purchased from the Zebrafish International Resource Center (ZIRC, Eugene, Oreg.). Embryos were manually dechorionated with forceps at 24 h post-fertilization (hpf), they were arrayed in 96-well plate, one embryo per well, and incubated with 100 μl of the indicated concentrations of the tested compounds at 28.5° C. for 24 h. DMSO was used as both carrier of drugs and control. After incubation, fish embryos were anesthetized with tricaine (0.02%), placed on slides and examined under an epifluorescence Nikon microscope equipped with DS-L1 Nikon digital camera. Phenotypic changes were evaluated by two different observers.

To further evaluate the in vivo antiangiogenic activity of AD0157, an assay based on the transgenic (TG(fli1:EGFP)y1) zebrafish line were used. In these animals, enhanced green fluorescent protein (EGFP) expression is driven under the 15-kb promoter of the transcription factor friend leukaemia virus integration-1 (fli-1). This promoter is ubiquitously activated in endothelial cells along the complete embryo, young and adult zebrafish. This ubiquitous expression leads to green in vivo fluorescence in all endothelial cells, and permits observation of bright blood vessels at all stages of embryogenesis. As shown in FIG. 8, 25 μM AD0157 added to water reduced the number of caudal intersegmental vessels at late tail bud stages of Fli-EGFP transgenic zebrafish. This inhibitory effect was dose-dependent (table 2).

TABLE 2
Inhibition of zebrafish intersegmental vessels by AD0157

| Dose (μM) | Positive/total | % Inhibition |
|---|---|---|
| 0 | 0/30 | 0 |
| 5 | 5/20 | 25 |
| 10 | 12/25 | 48 |
| 25 | 17/20 | 85 |

1. A compound of general formula (I): 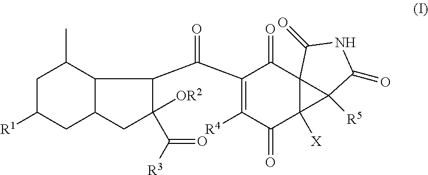
wherein R¹ is selected from the group consisting of —COOR^{a}, —CONHR^{a}, —CONR^{a}R^{b}, —COX, —CN, —C(═O)H, —CH₂OR^{a}, —CH₂X;
R^{a} and R^{b} are independently selected from the group consisting of hydrogen, substituted or unsubstituted acyl, substituted or unsubstituted alkyl, substituted or unsubstituted alkenyl, substituted or unsubstituted alkynyl, substituted or unsubstituted aryl, substituted or unsubstituted benzyl, alkali-metal, and sugar;
X is selected from the group consisting of hydrogen and halogen;
R² is selected from the group consisting of hydrogen, substituted or unsubstituted acyl, substituted or unsubstituted alkyl, substituted or unsubstituted alkenyl, substituted or unsubstituted alkynyl, substituted or unsubstituted aryl, substituted or unsubstituted benzyl, alkali-metal, and sugar;
R³, R⁴ and R⁵ are independently selected from the group consisting of hydrogen, substituted or unsubstituted alkyl, substituted or unsubstituted alkenyl and substituted or unsubstituted alkynyl;
or a pharmaceutically acceptable salt, stereoisomer, or solvate thereof.

2. The compound according to claim 1, wherein R¹ is selected from —COOR^{a}, —CONHR^{a}, —CONR^{a}R^{b}, more preferably it is —COOR^{a}.

3. The compound according to of claim 2, wherein R¹ is —COOH or a salt thereof.

4. The compound according to claim 1, wherein R² is hydrogen.

5. The compound according to claim 1, wherein R³ is substituted or unsubstituted alkenyl, preferably unsubstituted alkenyl.

6. The A compound according to claim 5, wherein R³ is 1-propenyl.

7. The compound according to claim 1, R⁴ is substituted or unsubstituted alkenyl, preferably unsubstituted alkenyl.

8. The compound according to claim 7, wherein R⁴ is 1-propenyl.

9. The compound according to claim 1 which is AD0157. 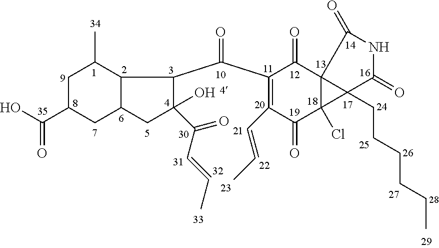 [SMILES: C/C=C/C(=O)C1(O)CC2CC(C(=O)O)CC(C)C2C1C(=O)C1=C(/C=C/C)C(=O)C2(Cl)C3(CCCCCC)C(=O)NC(=O)C23C1=O]
or a pharmaceutically acceptable salt, stereoisomer or solvate or thereof.

10. A process for the preparation of a compound of formula general (I) as defined in claim 1, or a pharmaceutically acceptable salt, stereoisomer, or solvate thereof, wherein the process comprises the step of isolating a compound of formula (I) above from a culture broth of a microorganism.

11. The process according to claim 10, wherein the microorganism is a fungal strain, more preferably a Paraconiothyrium sp.

12. The process according to claim 11, wherein the fungal strain is Paraconiothyrium sp. HL-78-gCHSP3-B005 which has been deposited in the Colección Española de Cultivos Tipo at the Universidad de Valencia, Spain under the accession number CECT 20841.

13. A pharmaceutical composition comprising at least one compound of general formula (I) as defined in claim 1, or a pharmaceutically acceptable salt, stereoisomer, or solvate thereof and a pharmaceutically acceptable excipient.

14. A method for inhibiting angiogenesis in a subject comprising administering to the subject a compound of general formula (I) as defined in claim 1, or a pharmaceutically acceptable salt, stereoisomer, or solvate thereof.

15. The method according to claim 14, for the treatment and/or prophylaxis of human diseases caused by a pathological angiogenesis process.

16. The method according to claim 15, wherein the human disease is selected from the group consisting of cancer, ocular diseases, psoriasis, hemangiomas, arthritis, endometriosis, and atherosclerosis.

17. The method according to claim 16, wherein the human disease is ocular disease, and the ocular disease is selected from the group consisting of diabetic and non-diabetic retinopathy, prematurity retinopathy and macular degeneration.

18. The method according to claim 16, wherein the human disease is ocular disease, and the arthritis is selected from the group consisting of osteoarthritis, rheumatoid arthritis, polyarthritis, gouty arthritis, lupus-associated arthritis, psoriasis-associated arthritis, and rheumatoid arthritis.
